(19) United States
(12) Patent Application Publication
(10) Pub. No.: US 2006/0206085 A1
(43) Pub. Date: Sep. 14, 2006

(54) Men's reuseable underwear with built-in absorbant panels

(75) Inventors: Chris Gegelys, Chester, NJ (US); Daniel M. Sivilich, Freehold, NJ (US)

Correspondence Address: EPSTEIN DRANGEL BAZERMAN & JAMES, LLP, 60 EAST 42ND STREET, SUITE 820, NEW YORK, NY, 10165, US

(73) Assignee: Humanicare International, Inc.

(21) Appl. No.: 11/078,556

(22) Filed: Mar. 10, 2005

(51) Int. Cl. A61F 13/15

(52) U.S. Cl. 604/385.14; 604/395; 604/396

(57) ABSTRACT

The underwear is fabricated from washable fabric formed into a pant. The pant has a front, a seat, a waistband and leg openings. The front includes a first portion having an inner flap with an edge and a second portion having an outer flap with an edge. The flaps are situated in overlapping relation, sewn to the pant front at spaced locations, to define a normally closed fly. First and second panels are provided. Each of the panels includes a liquid absorbent layer and a liquid barrier layer. The panels are attached to the pant front, on opposite sides of the fly, with the edge of the first panel substantially co-extensive with the edge of the inner flap. The edge of the second panel may be substantially aligned with the edge of the outer flap, or spaced a short distance therefrom, so as to form a substantially continuous liquid absorbent layer across the garment front.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO A “SEQUENCE LISTING”, A TABLE, OR A COMPUTER PROGRAM LISTING APPENDIX SUBMITTED ON COMPACT DISC

Not Applicable

### BACKGROUND OF THE INVENTION

1. Field of the Invention

The present invention relates to men's reusable undergarments of the type having a “fly” opening to permit withdrawal of the penis for urination and more particularly undergarments including loose fitting “boxer” shorts that include post void dribbling absorbent panels providing continuous protection across the front of the garment, without interfering with the operation of the “fly” opening.

2. Description of Prior Art Including Information Disclosed Under 37 CFR 1.97 and 1.98

The process of male urination is such that urine is excreted from the body via the urethra in the male penis. Upon completion, the sphincter muscle contracts shutting off the flow from the bladder. When that occurs, a small amount of residual urine is usually left in the urethra. This additional urine is often dispensed with by manually shaking the penis.

However, since the penis is extremely elastic, a few unexpected drops of residual urine may be excreted at a later time simply from penile movement. These droplets are usually absorbed by underwear to some extent, but due to the generally nonabsorbent nature of the fabric from which the underwear is made, they can also wick through the underwear and into the wearer's outer clothing (trousers, etc.). The wet spot can be visible on the outer clothing, especially if it is lightly colored. This can be annoying and is often perceived by most cultures as embarrassing. Further, once the wet spot on the outer clothing dries, an unsightly stain may remain.

A solution to this problem is to prevent the urine from wicking into the wearer's outer clothing. This can be accomplished in a variety of ways seen in prior art. Once solution is to form the crotch area of the undergarment itself of multilayer material with enhanced moisture management capacity, such as is disclosed in U.S. Pat. No. 5,210,882, entitled “Undergarment with Combination of Loose Fitting Seat, Upper Abdomen and Leg Areas and Crotch Area” issued to Moretz et al. on May 18, 1993. However, the undergarments disclosed in some of the embodiments of Moretz U.S. Pat. No. 5,210,882 have a tight fitting crotch area made of several layers and may be uncomfortable to wear.

U.S. Pat. No. 5,291,617 entitled “Moisture Management Garment” issued Mar. 8, 1994 to Moretz et al. and U.S. Pat. No. 6,782,557 entitled “Undergarment with Polymer Coated Fabric Layer For Protection Against Incontinence” issued Aug. 31, 2004 to Feder each teach garments with absorbent front portions that absorb moisture. However, those structures interfere with the function of the fly opening.

U.S. Pat. No. 5,217,782, entitled “Multi-Layer Moisture Management Fabric” issued to Moretz on Jun. 8, 1993 and U.S. Pat. No. 5,906,876 entitled “Absorbent Fabric and Undergarments Incorporating Same” issued to Conway on May 25, 1999 each teach a fabric for such an undergarment. However, neither of those fabrics has resulted in garments that provide the necessary protection across the entire front of the garment, which do not interfere with the function of the fly and at the same time are comfortable to wear.

The most common solution is to insert an absorbent disposable pad into the underwear such as disclosed in Moretz U.S. Pat. No. 5,210,882, noted above, wherein the pad is received in a pouch, and U.S. Pat. No. 4,589,877 entitled “Male Incontinence Device” issued to Sivilich on May 20, 1986 where an absorbent shield is adhesively attached to the undergarment. However, disposability becomes an issue, in that the pad or shield must be removed from the undergarment before washing. Moreover, such disposable articles tend to be thick and uncomfortable to wear and are not wide enough to provide protection across the entire front of the garment.

In the present invention, a reusable undergarment is provided with washable absorbent panels that are “permanently” fixed to the undergarment. The panel(s) are designed to absorb residual urine and and at the same time to provide a barrier to prevent it from wicking to the outer clothing.

Many of the styles of men's underwear have a front “fly” opening to allow for easy withdrawal of the penis from the underwear for urination. This fly opening is typically formed of overlapping flaps of the fabric from which the underwear is made. The flaps are sewn at the top and bottom creating a flexible aperture. Obviously, attaching an absorbent panel across the front of such a garment or retaining an absorbent pad or shield in the crotch area, as taught by the prior art, would cover the fly and seal it closed, rendering it non-functional.

In general, those problems are avoided in the present invention by utilizing two separate absorbent panels. The panels are attached to the front of the undergarment, on opposite sides of the fly opening, in a manner which provides continuous protection across the entire front of the undergarment. This allows the penis to be withdrawn and put back into the wearer's underwear through the fly opening, but still provide protection from embarrassing wetness.

The present invention is particularly useful for loose fitting undergarments such as “boxer” shorts that are typically made of a thin woven fabric. The fabric is usually not very absorbent and therefore transfers urine wetness quickly to the wearer's outer garment. However, the present invention is also applicable to tighter fitting undergarments, such as briefs.

It is, therefore, a prime object of the present invention to provide men's reusable underwear with built-in absorbent panels.

It is another object of the present invention to provide men's reusable underwear with built-in absorbent panels in which the fly opening is fully functional but, at the same time, full protection is provided.

It is still another object of the present invention to provide men's reusable underwear with built-in absorbent panels which protect the entire front of the garment from transmission of wetness resulting from post void dribbling.

It is still another object of the present invention to provide men's reusable underwear with built-in absorbent panels that looks and feels like conventional underwear.

It is still another object of the present invention to provide men's reusable underwear with built-in absorbent panels particularly suited for use in “boxer” shorts type underwear.

### BRIEF SUMMARY OF THE INVENTION

In accordance with one aspect of the present invention, reusable underwear for men is provided. The underwear is fabricated from washable fabric formed into a pant. The pant has a front, a seat, a waistband and leg openings. The front includes a first portion having an inner flap with an edge and a second portion having an outer flap with an edge. Means are provided for connecting the flaps in overlapping relation, at spaced locations, to define a normally closed fly. A first panel having an edge is provided. A second panel having an edge is provided. Each of the panels includes a liquid absorbent layer and a liquid barrier layer. Means are provided for attaching the first and second panels to the pant front, on opposite sides of the fly, with the edge of the first panel substantially co-extensive with the edge of the inner flap and the edge of the second panel substantially aligned with the edge of the outer flap, so as to form a substantially continuous liquid absorbent layer across the garment front.

The liquid absorbent layer of each of the panels preferably includes a knit cotton or cotton blend material. However, rayon, hydrophilic polyester, hydrophilic polypropylene, hydrophilic polyethylene or multiple fiber blends can also be utilized.

The liquid absorbent layer is preferably laminated to the liquid barrier layer. It also preferably has antimicrobial properties.

The liquid barrier layer is preferably moisture vapor permeable.

The liquid barrier layer preferably comprises urethane. The urethane is in the form of a thin film. The film is preferably less than 0.002″ thick.

The underwear preferably takes the form of a loose fitting garment such as boxer shorts.

Preferably, the panels are sewn to the pant front using thread. The panels may extend down the pant front and along the leg portions of the garment.

In accordance with another aspect of the present invention, reusable underwear for men is provided. The underwear is fabricated from washable fabric formed into a pant. The pant has a front, a seat, a waistband and leg openings. The front includes a first portion having an inner flap with an edge and a second portion having an outer flap with an edge. Means are provided for connecting the flaps in overlapping relation, at spaced locations, to define a normally closed fly. A first panel having an edge is provided. A second panel having an edge is provided. Each of the panels includes a liquid absorbent layer and a liquid barrier layer. Means are provided for attaching the first and second panels to the underwear front, on opposite sides of the fly, with the edge of the first panel substantially co-extensive with the edge of the inner flap and the edge of the second panel spaced from the edge of the outer flap, by a distance equal to or less than the with of the inner flap, so as to form a substantially continuous liquid absorbent layer across the garment front.

The liquid absorbent layer of each of the panels preferably includes a knit cotton or cotton blend material. However, rayon, hydrophilic polyester, hydrophilic polypropylene, hydrophilic polyethylene or multiple fiber blends may also be used.

The liquid absorbent layer is preferably laminated to the liquid barrier layer. It also preferably has antimicrobial properties.

The liquid barrier layer is preferably moisture vapor permeable.

The liquid barrier layer preferably comprises urethane. The urethane is in the form of a thin film. The film is preferably less than 0.002″ thick.

The underwear preferably takes the form of a loose fitting garment such as boxer shorts.

Preferably, the panels are sewn to the pant front using thread. The panels may extend down the pant front and along the leg portions of the garment.

### BRIEF DESCRIPTION OF THE SEVERAL VIEWS OF DRAWINGS

To those and to such other objects that may hereinafter appear, the present invention relates to reusable underwear for men with built-in absorbent panels, as described in detail in the following specification and recited in the annexed claims, taken together with the accompanying drawings, wherein like numerals refer to like parts, and in which:

FIG. 1 is a front elevation view of a ‘boxer” shorts type undergarment of the present invention, as it would appear on an individual;

FIG. 2 is a partially exploded view of the interior of the front of the undergarment of the present invention;

FIG. 3 is a vertical cross-sectional view of the front of the undergarment of the present invention;

FIG. 4 is a front elevation view of a first brief type style of undergarment of the present invention, as it would appear on an individual;

FIG. 5 is a front elevation view of a second brief type style of undergarment of the present invention, as it would appear on an individual;

FIG. 6 is a horizontal cross-sectional view of the front of a first preferred embodiment of the construction of an undergarment of the present invention;

FIG. 7 is a horizontal cross-sectional view of the front of a second preferred embodiment of the construction of an undergarment of the present invention; and

FIG. 8 is a front elevation view of a second preferred version of the “boxer” shorts embodiment of the present invention.

### DETAILED DESCRIPTION OF THE INVENTION

FIGS. 1, 4 and 5 respectively show the exterior of a loose fitting “boxer” shorts type, long-legged brief type and short-legged brief type versions of undergarments of the present invention. In each case, the exterior of the undergarment is virtually indistinguishable in appearance from the conventional counterpart. Thus, a man wearing the undergarment of the present invention will not be embarrassed because others will not be able to tell from the appearance of the exterior that he is wearing an undergarment with absorbent properties.

In each version of the present invention, the undergarment is fabricated from washable woven cotton or cotton blend fabric formed into a pant having a front 10, a seat (not shown), an elastic waistband 12 and leg openings 14. In the “boxer” shorts-type version of FIG. 1, the body and leg portions of the undergarment are relatively loose fitting. In the brief-type version, the body and leg portions are relatively tight fitting.

Pant front 10 includes a first portion 16 having an outer flap 18 with an edge 20. Front 10 also includes a second portion 22 having an inner flap 24 with an edge 26.

Means in the form of stitches 28, 30 are provided at vertically spaced location on pant front 10 for connecting outer flap 18 and inner flap 24 in overlapping relation to define a fly opening 32. Fly 32 is normally closed but can be opened to withdraw the penis for urination. In the “boxer” shorts version of FIG. 1, the fly is substantially a vertical line, off set slightly from the line vertically bisecting the garment. In the briefs of FIGS. 4 and 5, the fly is arcuate, and off set from the garment line bisecting the garment to a somewhat greater degree.

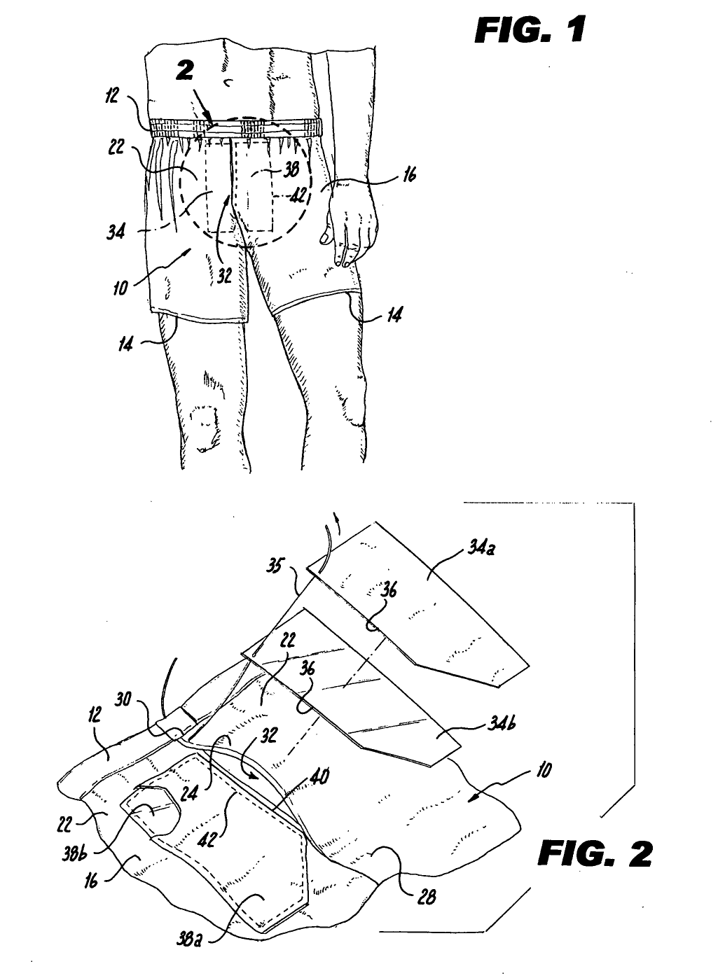

As illustrated in FIG. 2, a first absorbent panel 34 is provided with an edge 36. A second absorbent panel 38 is provided having an edge 40. Each of the absorbent panels 34, 38 is formed of a liquid absorbent layer 34a, 38a and a liquid barrier layer 34b, 38b, respectively.

Means, in the form of a thread 35, is provided for sewing first and second absorbent panels 34, 38 to the interior surface of the pant front 10, on opposite sides of the fly 32, along a stitch line 42. However, other conventional attachment techniques such as gluing, sonic welding, heat sealing and the like can be employed, if desired.

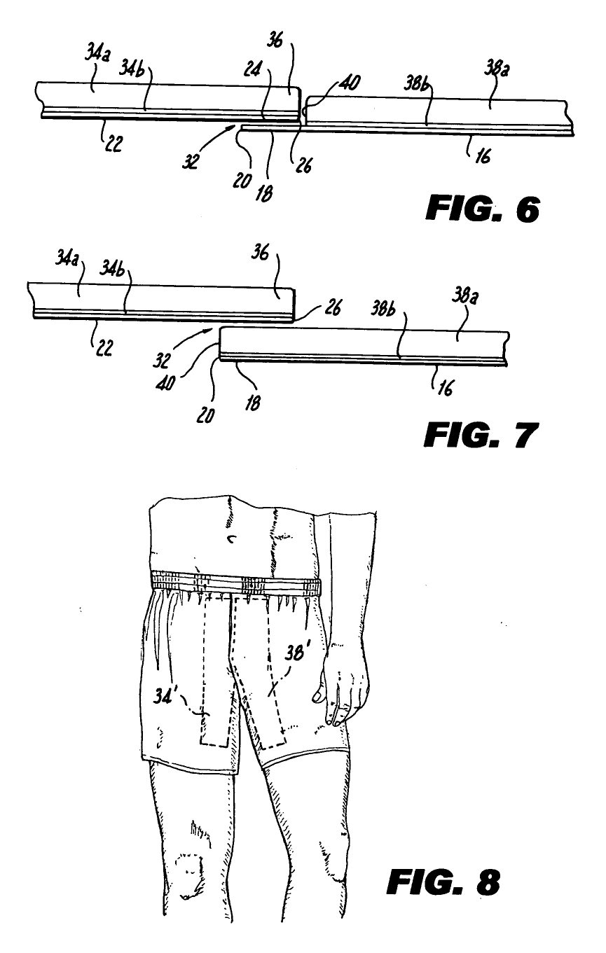

The panels are attached to the pant with edge 36 of first panel 34 substantially co-extensive with edge 26 of inner flap 24. The edge 40 of second panel 38 can be spaced a short distance from the edge 20 of outer flap 18, a distance equal to or less than the width of flap 24, and therefore be substantially aligned with edge 26 of inner flap 24, and proximate with edge 36 of first panel 34, when fly 32 is closed, as illustrated in FIG. 6, so as to form a substantially continuous liquid absorbent layer across front 10. Alternatively, edge 40 of second panel 38 can be substantially aligned with the edge 20 of the outer flap 18, as illustrated in FIG. 7, such that the panels 34, 38 will overlap in the fly area, and provide extra protection by forming a substantially continuous liquid absorbent layer across front 10, even when the fly 32 is partially open.

Panels 34, 38 can be constructed of a variety of materials that are absorbent, wicking and provide a moisture-proof barrier. In the preferred embodiment, each of the panels has two layers, being constructed of a liquid absorbent layer 34a, 38a and a liquid barrier layer 34b, 38b. The absorbent layer of the panel can be fabricate of a woven, knit or non-woven fabric made of absorbent fibers such as cotton but not limited to, rayon, cotton, hydrophilic polyester, hydrophilic polypropylene, hydrophilic polyethylene or multiple fiber blends. The liquid barrier layer can be, but is not limited to relatively thin films, foils, or other moisture proof substrates.

Preferably, the liquid absorbent layer 34a, 38a has antimicrobial properties. Those properties may be the result of treating the material of the liquid absorbent layer using an antimicrobial treatment. In the preferred embodiment, an advanced silane chemical treatment called AEGIS MICROBE SHIELD developed by AEGIS Environments, 2205 Ridgewood Drive, Midland Mich., 48642-5884 is utilized. That treatment involves an active ingredient that forms a colorless, odorless positively charged polymer which permanently bonds to the treated surface. It kills harmful microbes on contact, but does not affect normal skin flora.

In the preferred embodiment, an absorbent layer 34a, 38a of knit cotton or cotton blend fabric is laminated to a thin liquid barrier layer 34b, 38b of urethane film. The urethane film is preferably less than 0.002″ thick. The film acts as a liquid barrier, but has a high moisture vapor transmission rate. Thus, the wetness is contained, but it “breathable” to perspiration and evaporating urine. Strips of the laminate are cut and sewn by stitches 42 of thread 35 into the front area of an undergarment to either side of the fly opening 32. Thus the penis can be withdrawn from the pant for purposes of urination through the fly opening and returned through the same aperture.

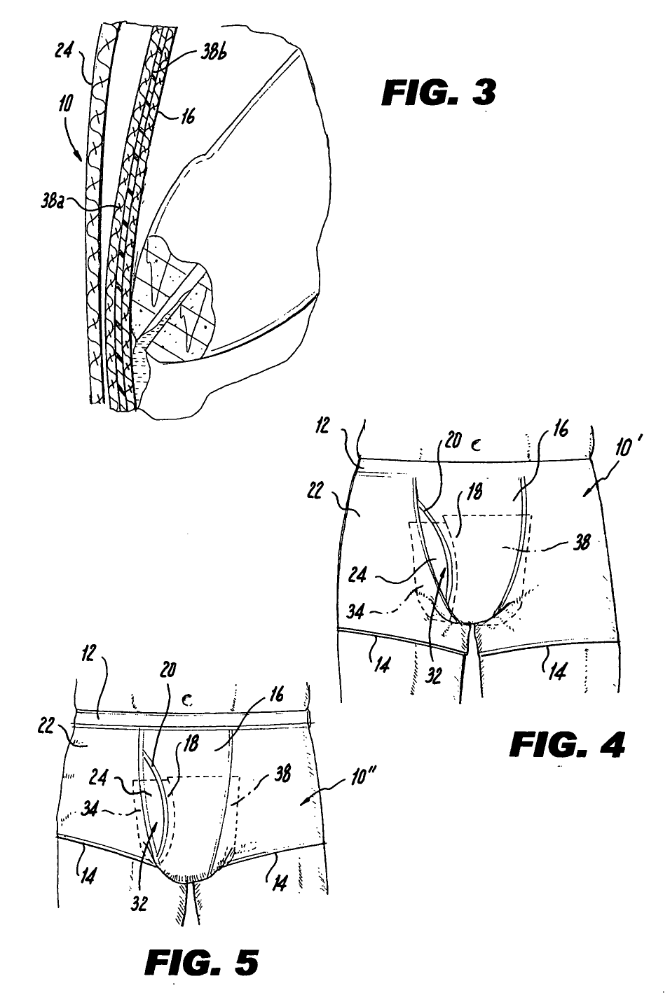

As illustrated in FIG. 3, any residual urine will be absorbed into the knit fabric absorbent layer of the panel where it is contained by the liquid barrier urethane film layer. Depending on the relative humidity of the environment, the water component of the urine can evaporate. The wearer's trousers are discretely protected from wetness.

FIG. 8 shows a second version of the “boxer” shorts preferred embodiment illustrated in FIG. 1. In the version there shown, absorbent panels 34′, 38′ extend downwardly beyond the crotch portion of the front of the pant, along the interior front surfaces of the leg sections, so as to provide additional protection in that area.

It will now be appreciated that the present invention relates to reusable men's underwear that is virtually indistinguishable in appearance from conventional garments of this type but which provides protection against wetting of outer clothing resulting from post void dribbling. That is accomplished through the use of absorbent panels located on either side of the fly opening. Each of the panels includes a liquid absorbent layer laminated to a liquid barrier layer made of a thin film such that the panel retains the liquid but is moisture vapor transmissive to permit evaporation.

While only a limited number of preferred embodiments have been disclosed herein for purposes of illustration, it is obvious that many variations and modifications could be made thereto. It is intended to cover all of those variations and modification that fall within the scope of the present invention, as set forth in the following claims:

We claim:

1. Reusable underwear for men fabricated from washable fabric formed into a pant comprising a front, a seat, a waistband and leg openings, said front comprising a first portion having an inner flap with an edge, a second portion having an outer flap with an edge, means for connecting said flaps in overlapping relation at spaced locations to define a normally closed fly opening, a first panel having an edge, a second panel having an edge, each of said panels comprising a liquid absorbent layer and a liquid barrier layer, means for attaching said first and second panels to said front, on opposite sides of said fly, with said edge of said first panel substantially co-extensive with said edge of said inner flap and said edge of said second panel substantially aligned with said edge of said first panel so as to form a substantially continuous liquid absorbent layer across said pant front.

2. The underwear of claim 1 wherein said edge of said second panel is spaced from said edge of said outer flap by a given distance.

3. The underwear of claim 2 wherein said given distance is equal to or less that the width of said inner flap.

4. The underwear of claim 1 wherein said liquid absorbent layer comprises cotton or a cotton blend.

5. The underwear of claim 1 wherein said liquid barrier layer comprises urethane.

6. The underwear of claim 5 wherein said urethane comprises a thin film.

7. The underwear of claim 6 wherein said film is less than 0.002″ thick.

8. The underwear of claim 1 wherein said liquid absorbent layer is laminated to said liquid barrier layer.

9. The underwear of claim 1 wherein said pant comprises relatively loose fitting “boxer” shorts.

10. The underwear of claim 1 wherein said pant comprises relatively snug fitting briefs.

11. The underwear of claim 1 wherein said attaching means comprises thread.

12. The underwear of claim 1 wherein said liquid barrier layer is moisture vapor permeable.

13. The underwear of claim 1 wherein said liquid absorbent layer comprises rayon.

14. The underwear of claim 1 wherein said liquid absorbent layer comprises hydrophilic polyester.

15. The underwear of claim 1 wherein said liquid absorbent layer comprises hydrophilic polypropylene.

16. The underwear of claim 1 wherein said liquid absorbent layer comprises hydrophilic polyethylene.

17. The underwear of claim 1 wherein said liquid absorbent layer comprises multiple fiber blends.

18. The underwear of claim 1 wherein said liquid absorbent layer has antimicrobial properties.

19. The underwear of claim 1 wherein said pant has leg portions and wherein said panels extend down said leg portions.

20. Reusable underwear for men fabricated from washable fabric formed into a pant comprising a front, a seat, a waistband and leg openings, said front comprising a first portion having an inner flap with an edge, a second portion having an outer flap with an edge, means for connecting said flaps in overlapping relation at spaced locations to define a normally closed fly opening, a first panel having an edge, a second panel having an edge, each of said panels comprising a liquid absorbent layer and a liquid barrier layer, means for attaching said first and second panels to said front, on opposite sides of said fly, with said edge of said first panel substantially co-extensive with said edge of said inner flap and said edge of said second panel substantially co-extensive with said edge of said outer flap, so as to form a substantially continuous liquid absorbent layer across said pant front.

21. The underwear of claim 20 wherein said liquid absorbent layer comprises cotton or a cotton blend.

22. The underwear of claim 21 wherein said liquid barrier layer comprises urethane.

23. The underwear of claim 22 wherein said urethane comprises a thin film.

24. The underwear of claim 23 wherein said film is less than 0.002” thick.

25. The underwear of claim 20 wherein said liquid absorbent layer is laminated to said liquid barrier layer.

26. The underwear of claim 20 wherein said pant comprises relatively loose fitting “boxer” shorts.

27. The underwear of claim 20 wherein said pant comprises relatively snug fitting briefs.

28. The underwear of claim 20 wherein said attaching means comprises thread.

29. The underwear of claim 20 wherein said liquid barrier layer is moisture vapor permeable.

30. The underwear of claim 20 wherein said liquid absorbent layer comprises rayon.

31. The underwear of claim 20 wherein said liquid absorbent layer comprises hydrophilic polyester.

32. The underwear of claim 20 wherein said liquid absorbent layer comprises hydrophilic polypropylene.

33. The underwear of claim 20 wherein said liquid absorbent layer comprises hydrophilic polyethylene.

34. The underwear of claim 20 wherein said liquid absorbent layer comprises multiple fiber blends.

35. The underwear of claim 20 wherein said liquid absorbent layer has antimicrobial properties.

36. The underwear of claim 20 wherein said pant has leg portions and wherein said panels extend down said leg portions.
